(19) United States
(12) Patent Application Publication
(10) Pub. No.: US 2022/0165422 A1
(43) Pub. Date: May 26, 2022

(54) DEVICE FOR GENERATING A MEDICAL KNOWLEDGE MODEL, MEDICAL KNOWLEDGE MODEL SYSTEM FOR THE FORMALIZED APPLICATION OF MEDICAL KNOWLEDGE, PROCESS FOR GENERATING A MEDICAL KNOWLEDGE MODEL, PROCESS FOR THE FORMALIZED APPLICATION OF MEDICAL KNOWLEDGE AND COMPUTER PROGRAM

(71) Applicant: Drägerwerk AG & Co. KGaA, Lübeck (DE)

(72) Inventors: Frank FRANZ, Lübeck (DE); Simon GISCH, Lübeck (DE); Hinrich ALTHOFF, Lübeck (DE)

(21) Appl. No.: 17/532,616

(22) Filed: Nov. 22, 2021

(30) Foreign Application Priority Data
Nov. 23, 2020 (DE) 10 2020 130 860.4

(51) Int. Cl. G16H 50/20; G06N 5/02

(52) U.S. Cl. CPC G16H 50/20; G06N 5/022

(57) ABSTRACT

A device generating a medical knowledge model, a medical knowledge model system for a formalized application of medical knowledge, a process generating a medical knowledge model, a process for the formalized application of medical knowledge, and a computer program are provided. The device (10) for generating a medical knowledge model includes a processing device (11), which is configured for providing at least one element from a standardized nomenclature for a user, for adding the at least one element from the standardized nomenclature to the medical knowledge model according to a selection of the user, and for storing a logic or a rule with regard to the at least one element concerning at least one element parameter according to a definition of the user. The device (10) includes one or more interfaces (12), which are configured for providing the medical knowledge model and for receiving a user input.

### CROSS REFERENCE TO RELATED APPLICATIONS

This application claims the benefit of priority under 35 U.S.C. § 119 of German Application 10 2020 130 860.4, filed Nov. 23, 2020, the entire contents of which are incorporated herein by reference.

### TECHNICAL FIELD

The present invention pertains to a device for generating a medical knowledge model, to a medical knowledge model system for the formalized application of medical knowledge, to a process for generating a medical knowledge model, to a process for the formalized application of medical knowledge, and to a computer program, especially, but not exclusively, to a concept for generating, applying and/or expanding a medical knowledge model by means of a logic or a rule according to a definition of the user.

### TECHNICAL BACKGROUND

Distributed systems of electrical medical devices and clinical IT systems at a clinical work area are more and more becoming increasingly important because it is expected that an added value can be obtained in the treatment and/or monitoring of patients due to integration at the data and function level. At the same time, the successful treatment of critical patients is becoming increasingly more complicated, and an increasing number of guidelines and more complicated guidelines are being issued by expert associations to do this justice. In addition, the care ratio is becoming increasingly worse, on the one hand, in order to save staff costs and, on the other hand, because qualified staff is becoming increasingly difficult to find and to retain.

Solutions for decision support, automatic monitoring or remote control may improve the diagnosis, therapy and care. Staff workload may also be reduced. There are automatic rule-based systems for decision support, for example, SmartSonar Sepsis, SmartCare, drug regulation, as well as rule-based closed-loop systems. The rules and workflows, which contain the clinical knowledge for the systems, are, for example, represented via an Arden Syntax or proprietary formats. In addition to decision trees, additional model-based approaches and artificial intelligence methods are used.

For example, there are already the following scientific publications that pertain to this topic:
Taqdir Ali; Maqbool Hussain; Wajahat Ali Khan; Muhammad Afzal; Byeong Ho Kang; Sungyoung Lee. Arden syntax studio: Creating medical logic module as shareable knowledge, 2014 IEEE International Symposium on Innovations in Intelligent Systems and Applications (INISTA) Proceedings, Jun. 23-25, 2014; this scientific publication pertains to the Arden Syntax;
Scheepers-Hoeks A M, Grouls R J, Neef C, Korsten H H. Strategy for implementation and first results of advanced clinical decision support in hospital pharmacy practice. Stud. Health Technol. Inform., 2009; 148:142-148; this scientific publication pertains to decision trees;
AiD Klinik, https://www.dosing-gmbh.de/produktloesungen/aidklinik-2/, shows a drug information system with drug catalog and current knowledge data;
Stojadinovic A, Bilchik A, Smith D, Eberhardt J S, Ward E B, Nissan A et al. Clinical decision support and individualized prediction of survival in colon cancer: Bayesian belief network model. Ann Surg Oncol, 2013; 20 (1): 161-174; this scientific publication pertains to model-based approaches;
Jalali A, Bender D, Rehman M, Nadkanri V, Nataraj C. Advanced analytics for outcome prediction in intensive care units. Conf Proc IEEE Eng Med Biol Soc. 2016; 2016: 2520-2524; this scientific publication pertains to artificial intelligence methods; and
Tenorio J M, Hummel A D, Cohrs F M, Sdepanian V L, Pisa I T, de Fatima Marin H. Artificial intelligence techniques applied to the development of a decision support system for diagnosing celiac disease. Int. J. Med. Inform. 2011, 80 (11): 793-802; this scientific publication also pertains to artificial intelligence methods.

For clinical staff, it is often difficult using current methods to formulate medical knowledge that can be executed automatically for automation, since the medical knowledge is, for example, often not formalized from textbooks and/or publications. A Syntax, for example, Arden, is difficult to grasp and difficult to understand for clinical staff with relevant medical knowledge. Frequently, no knowledge about systematic risk management is present here. The other way around, technical staff usually lack clinical knowledge.

On the one hand, there is a lack of transparency in the application because black box systems with third-party contents inspire little confidence, and there are high regulatory obstacles. The technique is based on modeled or proprietary information in order to identify elements, which are used in the modeling of knowledge, and not on open standards. Updated regulations can only be delivered by an updated version of the software, which causes costs for the manufacturer of the Clinical Decision Support Systems, CDSS, and makes it very difficult to adapt to the new state of the knowledge in a timely manner. There are tools, though, which show approaches to solve this problem, for example, d3web with DiaFlux, e.g., https://www.d3web.de/Wiki.jsp?page=Doc%20Diaflux, but they have not solved the delivery problem.

### SUMMARY

Against this background, there is therefore a need to provide medical knowledge in a formalized manner in a simple and reliable way. This need is met by the device, system and process of the invention.

Exemplary embodiments are based on the core idea of generating a medical knowledge model in accordance with user specifications. At least one element from a standardized nomenclature can hereby be added, according to a selection by a user, to a medical knowledge model together with a logic or a rule with regard to the at least one element according to a definition of the user. This can make it possible to provide medical knowledge in a formalized manner in a simple and reliable manner. Exemplary embodiments may make available a tool, which can make it possible to model concentrated medical knowledge. A graphics editor, which may contain elements from routine clinical practice for modeling the knowledge, can be made available for this purpose. Among other things, elements may be physiological parameters, drugs, diagnoses, procedures, lab results, patient core data, fluid balances, allergies, results of clinical assessments, results of clinical expert opinions, results and/or scores but also settings on devices or roles of the clinical staff

Exemplary embodiments create a device for generating a medical knowledge model. The device comprises a processing device configured for providing at least one element from a standardized nomenclature for a user. The processing device is further configured for adding the at least one element from the standardized nomenclature to the medical knowledge model according to a selection of the user and for storing a logic or a rule with regard to the at least one element concerning at least one element parameter according to a definition of the user. Furthermore, the device comprises one or more interfaces which are configured for providing the medical knowledge model and for receiving a user input. Medical knowledge can hereby be provided in the medical knowledge model in a formalized manner in a simple and reliable manner.

The processing device may further be configured in this case for validating a consistency of the medical knowledge model. The consistency of the medical knowledge model can in this case be automatically validated. This can make possible a secure generation of a tested medical knowledge model.

The processing device is further configured according to the present invention for adding a digital signature to the medical knowledge model. The user can ensure by the signature that the medical knowledge model comes from a recognized qualified preparer, corresponds to a guideline and/or was not changed.

The processing device may be further configured in some exemplary embodiments for offering the user as a logic or as a rule at least one element from the group comprising physiological variables, settings, roles, mathematical and statistical operations, Boolean logic, an “if-then” case distinction and temporal criteria. In this case, the logic or the rule corresponding to the respective application requirements may be defined by the user in a precisely fitting manner for the respective application.

In other exemplary embodiments, the standardized nomenclature may comprise a medical guideline. As a result, the medical knowledge model may be even further improved and thus a treatment may be offered to the patient that is adapted to his needs.

Furthermore, the at least one medical element may comprise a measurement of a vital parameter of a patient. The treatment of the patient may be adapted even more individually to the patient and to the patient's current health status and/or to the patient's current course of therapy in this case.

Exemplary embodiments create, furthermore, a medical knowledge model system for the formalized application of medical knowledge. The medical knowledge model system may comprise the device. The processing device may be further configured for executing the logic or the rule with regard to the at least one element concerning the at least one element parameter from the standardized nomenclature, and for expanding the medical knowledge model by at least one additional element from the standardized nomenclature. The one or more interfaces may be further configured for providing the element. As a result, it can be made possible to apply and/or to expand the medical knowledge model in a rapid, simple and reliable manner. The medical knowledge model may also be updated, for example, by expanding the medical knowledge model.

In other exemplary embodiments, the processing device may be further configured for generating output information during the execution of the logic or of the rule. The one or more interfaces may be further configured here for providing the output information. Information can be provided by the output information to the user for designing a therapy or a treatment of the patient.

Exemplary embodiments create, furthermore, a process for generating a medical knowledge model. The process comprises the provision of at least one element from a standardized nomenclature for a user, the addition of the at least one element from the standardized nomenclature to the medical knowledge model according to a selection of the user, and storage of a logic or of a rule with regard to the at least one element concerning at least one element parameter according to a definition of the user. This may make possible a simple and reliable provision of medical knowledge in the medical knowledge model in a formalized manner.

In some exemplary embodiments, the process may further comprise validation of the consistency of the medical knowledge model. To validate the consistency, the medical knowledge model may be checked for, among other things, circular dependencies or incomplete pathways. The consistency of the medical knowledge model can in this case be automatically validated and/or the medical knowledge model can be viewed by a clinical expert. A tested medical knowledge model can be generated as a result.

The process according to the present invention has, furthermore, an addition of a digital signature to the medical knowledge model. It can be ensured by means of the signature that the medical knowledge model comes from a recognized and qualified preparer, corresponds to a guideline and/or was not changed.

Exemplary embodiments create, furthermore, a process for the formalized application of medical knowledge. The process may comprise the process for generating a medical knowledge model. The process may further comprise the execution of the logic or of the rule with regard to the at least one element concerning the at least one element parameter from the standardized nomenclature, and expansion of the medical knowledge model by at least one additional element from the standardized nomenclature.

In this manner, the medical knowledge model can be applied and/or expanded in a rapid, simple and reliable manner. The expansion of the medical knowledge model may also comprise, for example, in addition to the addition of medical knowledge from a standardized nomenclature, an updating of the medical knowledge model.

Another exemplary embodiment is a computer program with a program code for carrying out one of the processes described herein, when the program code is executed on a computer, on a processor or on a programmable hardware component. A machine-readable data storage medium with such a program code is another exemplary embodiment.

Some examples of the devices and/or the processes will be explained in more detail below only as examples with reference to the attached figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a block diagram of an exemplary embodiment of a device for generating a medical knowledge model;

FIG. 1a is a view showing a concept of an editor for preparing rules for the example of Critical Care (central critical care);

FIG. 1b is a block diagram showing a concept of a DiaFlux Workflow & Requirement Specification;

FIG. 2 is a block diagram of an exemplary embodiment of a process for generating a medical knowledge model;

FIG. 3 is a block diagram of an exemplary embodiment of a process for generating a medical knowledge model; and

FIG. 4 is a block diagram of an exemplary embodiment of a process for the updated application of medical knowledge.

### DESCRIPTION OF PREFERRED EMBODIMENTS

Referring to the drawings, different examples will now be described in more detail with reference to the attached figures. The thicknesses of lines, layers and/or areas may be exaggerated for illustration in the figures.

Further examples may cover modifications, equivalents and alternatives, which fall within the scope of the disclosure. Identical or similar reference numbers refer in the entire description of the figures to identical or similar elements, which may be implemented identically or in a modified form in a comparison with one another, while they provide the same function or a similar function.

It is apparent that if an element is described as being “connected” or “coupled” to another element, the elements may be connected or coupled directly or via one or more intermediate elements. When two elements A and B are combined with the use of an “or,” this shall be defined as that all possible combinations are disclosed, i.e., only A, only B, as well as A and B, unless this is defined explicitly or implicitly as being otherwise. An alternative wording for the same combinations is “at least one of A and B” or “A and/or B.” The same applies, mutatis mutandis, to combinations of more than two elements.

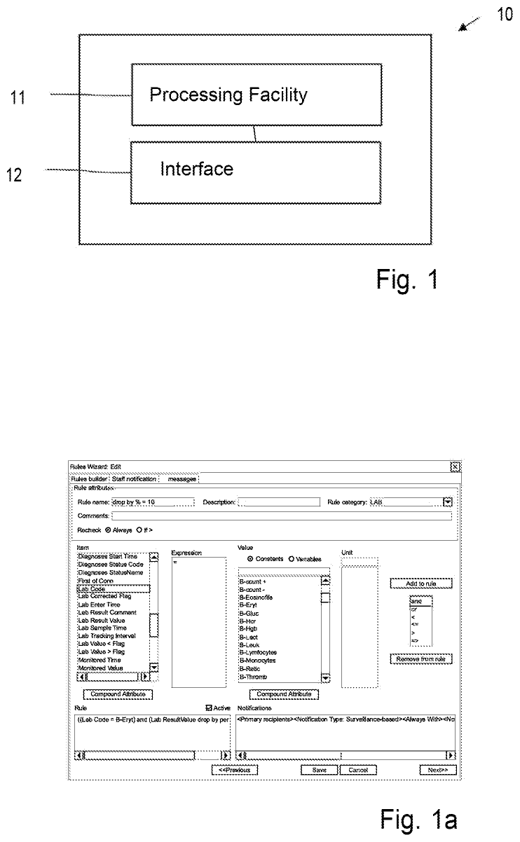

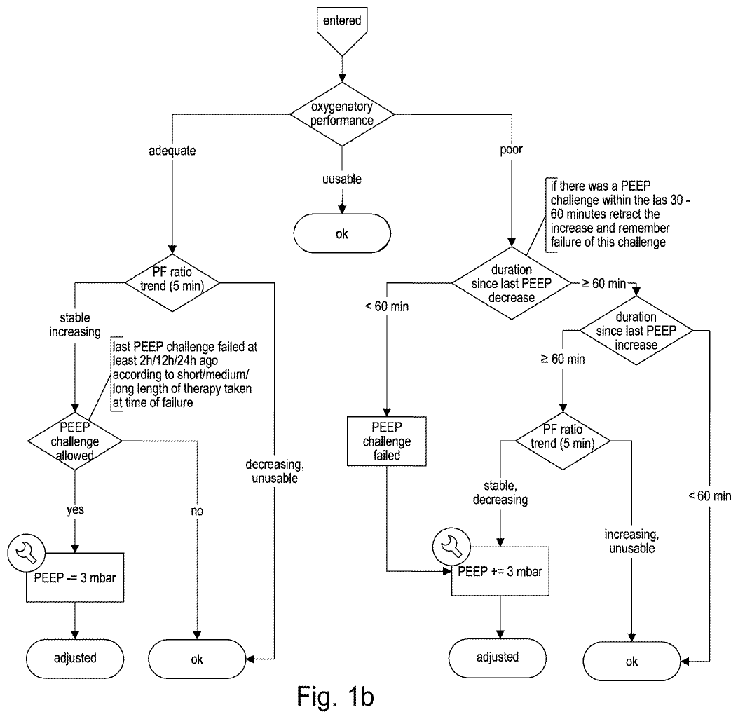

FIG. 1 shows a block diagram of an exemplary embodiment of a device 10 for generating a medical knowledge model. The medical knowledge model comprises a logic or a rule, which refers to elements from a standardized nomenclature. Medical knowledge can be provided in a formalized manner in a simple and reliable manner by means of the medical knowledge model. Workflows can be better structured, as a result, for example, in case of a monitoring and/or treatment of patients in hospitals. The device 10 comprises a processing device 11. The processing device 11 may be configured in the form of a processor, especially in the form of a system on a chip.

The processing device 11 may comprise in exemplary embodiments one or more freely selectable controllers, microcontrollers, network processors, processor cores, such as Digital Signal Processor Cores (DSPs), programmable hardware components, etc. Exemplary embodiments are not limited here to a defined type of processor core. Freely selectable processor cores or even a plurality of processor cores or microcontrollers are conceivable for implementing a processing device 11. The processing device 11 may be implemented in an integrated form with other devices, for example, in a control unit, which additionally also comprises one or more other functions. A processing device 11 may be embodied in exemplary embodiments by a processor core, by a computer processor core (CPU=Central Processing Unit), by a graphics processor core (GPU=Graphics Processing Unit), by an application-specific integrated circuit core (ASIC=Application-Specific Integrated Circuit), by an integrated circuit (IC=Integrated Circuit), by an one-chip system core (SOC=System on Chip), by a programmable logic element or a field-programmable gate array with a microprocessor (FPGA=Field Programmable Gate Array) as a core of the component or of the components.

The processing device 11 is configured for providing at least one element from a standardized nomenclature for a user. Among other things, the elements may be, for example, physiological parameters, drugs, diagnoses, procedures, lab results, patient core data, fluid balances, allergies, results of clinical assessments and scores, but also settings on devices or roles of clinical staff. In this case, the elements in the modeling tool may be based on open standards, terminologies and nomenclatures. Quality criteria, which may at times be contained in implementations compliant with the standards, may be assigned to the elements. Examples of open standards, terminologies and nomenclatures, include the following, which are incorporated herein by reference:
11073-20701-2018—IEEE Standard—Health informatics point of care medical device communication—Part 20701, Service-Oriented Medical Device Exchange Architecture and Protocol Binding, https://standards.ieee.org/standard/11073-20701-2018.html; SNOMED CT, Systematized Nomenclature of Medicine, Clinical Terms, http://www.snomed.org, https://confluence.ihtsdotools.org/display/DOC; international statistical classification of diseases and related health problems, https://www.dimdi.de/dynamic/de/klassifikationen/icd/icd-10-gm/.

The processing device 11 is, furthermore, configured for adding the at least one element from the standardized nomenclature to the medical knowledge model according to a selection of the user. The processing device 11 is further configured for storing a logic or a rule with regard to the at least one element concerning at least one element parameter according to a definition of the user. Examples of the element parameter may be an upper limit and/or lower limit for a respiratory rate, a blood pressure, a heart rate of the patient, a dosage rate for an administration of a drug to the patient or a duration since conclusion of a surgery.

The device 10 comprises, furthermore, one or more interfaces 12, which are coupled with the processing device 11 and which are configured for providing the medical knowledge model and for receiving a user input. The one or more interfaces 12 may be configured, for example, in the form of a machine interface or in the form of a software interface.

The one or more interfaces 12 may be configured in exemplary embodiments as typical interfaces for communication in networks or between network components or medical devices. For example, this interface 12 may be configured in exemplary embodiments by corresponding contacts. It may also be configured in exemplary embodiments as separate hardware and comprise a memory, which stores the signals to be sent or the signals received at least temporarily. The one or more interfaces 12 may be configured for receiving electrical signals, for example, as a bus interface, as an optical interface, as an ethernet interface, as a wireless interface, as a field bus interface, etc. It may, in addition, be configured in exemplary embodiments for wireless transmission and comprise a radio front end as well as corresponding antennas. Input devices and/or output devices, for example, a display screen, a keyboard, a mouse, may also be connected via the one or more interfaces 12, in order to detect user inputs and/or to make outputs possible.

The processing device 11 may be further configured for validating a consistency of the medical knowledge model. A plausibility of the rules and/or a data quality of the elements and/or element parameters may be examined here. Furthermore, the medical knowledge model may be checked for, among other things, circular dependencies or incomplete pathways. A circular dependence is present if reference is again made to a previous object in a series of references, in a referencing series or a series of referrals to an object located further back, so that a closed loop is formed. Circular dependencies may in many cases lead to contradictions or to unsolvable problems. It is checked whether the logic has undefined areas during the checking for incomplete pathways. The medical knowledge model and/or the consistency thereof may optionally be viewed and/or checked by a clinical expert. The clinical expert may be, for example, medically trained staff as well as physicians, nurses and/or health care staff. The tool or medical knowledge model may contain a checking of the modeled knowledge. Further, a validity and integrity can be checked not only within a model. A plurality of models may be brought together here for the checking.

Furthermore, the processing device 11 is configured for adding a digital signature to the medical knowledge model. The knowledge model prepared in this manner may be provided with a digital signature. The preparers may do this themselves, for example, if they are correspondingly qualified. For example, manufacturers of medical devices, scientific institutes, scientific medical societies, designated bodies or qualified third parties may release knowledge models by signature. The validity may then be checked by an executing system.

The processing device 11 may be further configured for offering to the user as a logic or as a rule at least one element from the group comprising physiological variables, settings, roles, mathematical and statistical operations, Boolean logic, an “if-then” case distinction and temporal criteria. The Boolean logic works with logical operators, for example, and, or, not, in order to define a logic. The “if-then” case distinction may make it possible to define a two-sided case distinction. An IF instruction may consist of a condition and two sequences of instructions. Here, one sequence of instructions may be defined for the case that the condition is met and one sequence of instructions may be defined for the other case. The condition may be, for example, that a value from a blood count of the patient is above a threshold value. If this condition is met, a drug is administered. Otherwise, the next measurement of the blood count is carried out in a predefined time interval. Temporal criteria make it possible to define time-dependent conditions, for example, to begin the administration of a drug only 12 hr. after a surgery.

The standardized nomenclature may comprise a medical guideline. Medical knowledge may be present, for example, in the form of coordinated guidelines from recognized scientific societies or from other accepted bodies. Medical guidelines are systematically developed determinations which physicians, dentists, members of other health professions and patients shall support in making their decisions about appropriate health care under specific clinical circumstances.

In this case, the at least one medical element may comprise a measurement of a vital parameter of a patient. The at least one medical element may comprise, for example, a measurement of the heart rate, of the blood pressure, of the oxygen saturation and/or of the respiratory rate as a measurement of a vital parameter.

An exemplary embodiment may pertain to a medical knowledge model system for the formalized application of medical knowledge. The medical knowledge model system comprises the device 10. The device 10 may have a configuration similar to the device 10 described in conjunction with FIG. 1. The processing device 11 may be further configured in this case for executing the logic or the rule with regard to the at least one element concerning the at least one element parameter from the standardized nomenclature. The processing device 11 may be further configured for generating output information during the execution of the logic or of the rule. The output information may comprise an instruction, which refers to a measurement, the presence of a measurement, and/or to a checking of a result of a measurement, which refers, for example, to blood values, respiratory rate, blood pressure, oxygen saturation and/or heart rate of the patient. Furthermore, the one or more interfaces 12 may be configured for providing the output information.

The processing device 11 may be further configured for expanding the medical knowledge model by at least one additional element from the standardized nomenclature. During the expansion of the medical knowledge model, for example, additional logical links, which pertain to the additional element, can be incorporated into the medical knowledge model. The additional elements may be, among other things, physiological parameters, drugs, diagnoses, procedures, lab results, patient core data, fluid balances, allergies, results of clinical assessments and scores, but also settings on devices or roles of the clinical staff The one or more interfaces 12 may be further configured for providing the element. In addition to adding medical knowledge from a standardized nomenclature, the expansion of the medical knowledge model may also comprise, for example, an updating of the medical knowledge model.

The exemplary embodiment shown in FIG. 1 may comprise one or more additional optional features.

FIG. 1a shows a concept of an editor for preparing rules using the example of Critical Care. This editor is used for generating a rule-based system for decision support. Formal knowledge, which medical staff does not possess in most cases, is necessary to generate a decision support system with this editor. FIG. 1b shows another concept of a DiaFlux Workflow & Requirement Specification. This concept shows a flow chart for oxygen output. A left pathway of the flow chart shows an adequate oxygen output and a right pathway of the flow chart shows a low oxygen output. These concepts have the drawback that under the circumstances, they do not make it possible for the users to themselves easily formalize medical knowledge. Vis-à-vis these concepts, the device being shown in FIG. 1 has the advantage that the medical knowledge can be formalized according to a definition of the user in a simple and reliable manner.

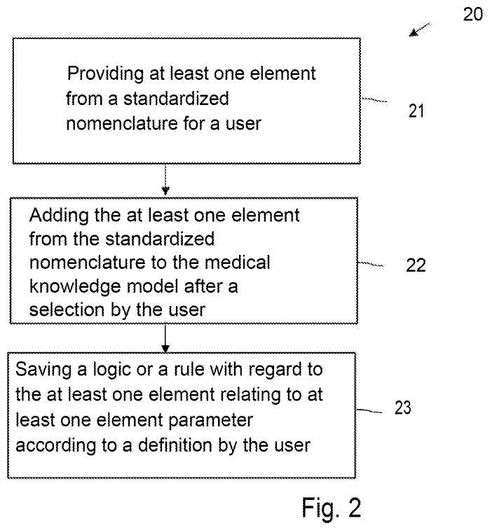

FIG. 2 shows a block diagram of an exemplary embodiment of a process 20 for generating a medical knowledge model. The process 20 comprises the provision 21 of at least one element from a standardized nomenclature for a user. The process 20 further comprises the addition 22 of the at least one element from the standardized nomenclature to the medical knowledge model according to a selection of the user. The process 20 further comprises the storage 23 of a logic or of a rule with regard to the at least one element concerning at least one element parameter according to a definition of the user.

Furthermore, the process 20 may comprise validation of the consistency of the medical knowledge model. Optionally, the medical knowledge model and/or the consistency thereof may be viewed and/or checked by a clinical expert. The validation of the consistency may also be carried out by the processing device 11, for example, on the basis of a predefined algorithm. The validation of the consistency may be carried out especially automatically.

The process 20 further comprises an addition of a digital signature to the medical knowledge model (not being shown in FIG. 2). The preparers may do this themselves, if they are correspondingly qualified. Moreover, for example, manufacturers of medical devices, scientific institutes, scientific medical societies, designated bodies or qualified third parties may release knowledge models by signature.

Exemplary embodiments may, moreover, be or pertain to a computer program with a program code for carrying out one or more of the above processes 20 when the computer program is executed on a computer, on a processor or on a programmable hardware component or on the processing device 11. Steps, operations or processes of different processes described above can be executed by programmed computers or processors. Examples may also cover program memory devices, e.g., digital memory media, which are machine-, processor- or computer-readable and code machine-executable, processor-executable or computer-executable programs of instructions stored on a non-transitory tangible medium. In particular, a computer readable storage medium or machine-readable data storage medium, particularly a tangible medium excluding signals, carrier waves, or other transitory signals with such a program code stored thereon is another exemplary embodiment, namely a non-transitory, machine-readable tangible data storage media with program code stored thereon.

The instructions carry out some steps or all the steps of the above-described processes or cause them to be executed. The program memory devices may comprise or be, e.g., digital memories (flash memories or solid state drive memories), magnetic storage media, for example, magnetic disks and magnetic tapes, hard drives or optically readable digital storage media—non-transitory, machine-readable tangible data storage media with program code stored thereon. Additional examples may also cover computers, processors or control units, which are programmed to execute the steps of the above-described processes, or (field)-programmable logic arrays ((F)PLAs=(Field) Programmable Logic Arrays) or (field)-programmable gate arrays ((F)PGA=(Field) Programmable Gate Arrays), which are programmed to execute the steps of the above-described processes.

The exemplary embodiment shown in FIG. 2 may comprise one or more additional optional features.

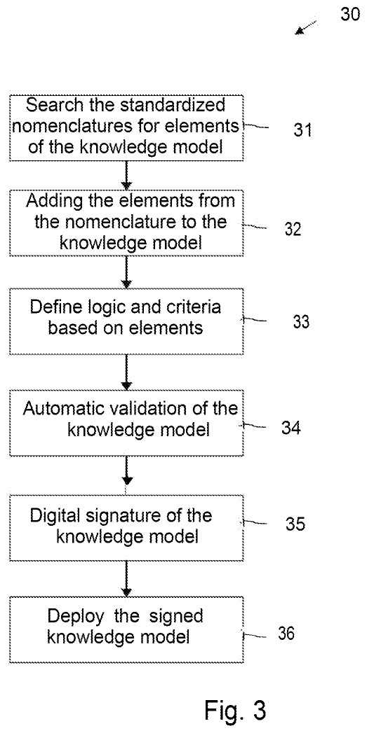

FIG. 3 shows a block diagram of an exemplary embodiment of another process 30 for generating a medical knowledge model. The process 30 may be embodied similar to the process 20 described in conjunction with FIG. 2. The process 30 may provide a tool, which can make it possible to model concentrated medical knowledge. A graphics editor, which may contain elements from the routine clinical practice for modeling the knowledge, may be made available for this purpose.

The process 30 may comprise a search 31 of the standardized nomenclature for elements of the knowledge model or of the medical knowledge model, which corresponds to the above-described provision 21 of at least one element from a standardized nomenclature for a user. The process 30 may further comprise an addition 32 of the elements from the nomenclature to the knowledge model. This corresponds to the above-described addition 22 of the at least one element from the standardized nomenclature to the medical knowledge model according to a selection of the user. The process 30 may further comprise a definition 33 of a logic and criteria based on the elements. For example, the definition 33 of the logic and criteria based on the elements may be carried out as described below. This corresponds to the storage 23 of a logic or of a rule with regard to the at least one element concerning at least one element parameter according to a definition of the user, which storage 23 was described on the basis of FIG. 2. The medical knowledge may be formalized, for example, with a Rule Editor using Drag & Drop as graphics editor. The Rule Editor may offer the possibility to select from physiological variables, settings, roles, mathematical (+, −, %, *, /, \hat{ }) and statistical operations (minimum, maximum, average), Boolean logic (AND, OR, NOR, XOR, NAND, . . . ), simple “if . . . then” case distinctions as well as temporal criteria for the modeling. An expansion to clinical results, complex calculations, signal characteristics may be possible.

The process 30 may comprise an automatic validation 34 of the knowledge model. The consistency of the generated knowledge model may be automatically validated hereby and/or the knowledge model may be checked for circular dependencies and/or incomplete pathways, among other things. The validation may be carried out by the processing device 11. Optionally, the model can be viewed and/or checked by a clinical expert. In this case, the process 30 comprises an addition 35 of a digital signature to the knowledge model. The knowledge model generated in this manner may be signed digitally. The signature may ensure that the knowledge model comes from a recognized qualified preparer, corresponds to a guideline and/or was not changed. The process 30 may further comprise a provision 36 of the signed knowledge model. Digitally signed knowledge models may be introduced into a Rule Engine configured in this manner in a networked clinical setting, e.g., be delivered, based on IEEE 11073 SDC (Service-oriented Device Connectivity), on a technical medical device, on a PC, centrally in the network or in the cloud. In case of an update of a guideline, a corresponding updated knowledge model may supersede the present knowledge model.

The IEEE 11073 SDC standard family defines a communication protocol for medical devices whose field of use is in the potentially critical patient care, e.g., in the operating room, in the intensive care unit, but in other areas of the hospital as well. It is part of the ISO/IEEE 11073 family of standards. IEEE 11073 SDC is based on the paradigm of service-oriented architecture, SOA. Introduction of SDC as a standard for the bidirectional communication of measured values and set points between medical devices may also open up possibilities of remote control.

According to one exemplary embodiment, the process 20, 30 may proceed as described below. The Deutsche Gesellschaft für Anästhesiologie and Intensivmedizin (German Society of Anesthesiology and Intensive Care Medicine), DGAI, may obtain a tool, a medical knowledge model or a medical knowledge model system in order to be able to model knowledge in a guideline “S3 guideline for the intensive medical care of cardiac surgery patients—Hemodynamic Monitoring and Coronary Circulation.” For example, the guideline from Habicher et al., S3 guideline for the intensive medical care of cardiac surgery patients, Hemodynamic Monitoring and Coronary Circulation,
https://www.awmf.org/uploads/tx_szleitlinien/001-0161_S3_Intensivmedizinische_Versorgung-Haemodynamisches-Monitoring_2018-06.pdf. The DGAI may resolve to implement at least those recommendations with a “Desired” grade (strong recommendation, Grade of Recommendation A).

The recommendations with a “Desired” grade may also be called strong recommendation or Grade of Recommendation A, “Empfehlungsgrad A” in German. The tool for modeling, the medical knowledge model or the medical knowledge model system may contain elements based on the nomenclature of the open 11073-10101 Standard “IEEE P11073-10101™ Standard for Health Informatics-Point-of-Care Medical Device Communication-Nomenclature,” BICEPS and OPS Version 2020.

The modeling is described as an example below on the basis of two recommendations from the guideline, which are modeled. According to one exemplary embodiment, pulse oximetry may be referred to as a core recommendation. Pulse oximetry is a process for noninvasive determination of the arterial oxygen saturation via the measurement of the light absorption or of the light remission during the transillumination of the skin. The pulse oximeter represents a spectrophotometer, which is especially optimized for this application. In addition, the device used may also be used for the simultaneous checking of the pulse rate. The guideline may state the following in this case: A continuous monitoring by means of pulse oximetry may detect an occurrence of clinically inapparent O₂ desaturations and may therefore be used as a continuous monitoring process.

In this case, a modeler may open in a tool a mechanism in order to search the nomenclature of an SDC standard. The modeler may in this case find an element for an SpO₂ measurement with the code 150456 and the reference MDC_PUL_OXIM_SAT_02. By Drag & Drop, the modeler may add the element to the model or to the medical knowledge model. In addition, the modeler may identify in a patient core data model a stay in the intensive care unit as an inclusion criterion (e.g., from the corresponding FHIR resource, FHIR—Fast Healthcare Interoperability Resource). Then, the modeler may, for example, add by a click a basic rule for checking or testing the presence of the measurement.

According to another exemplary embodiment, which may pertain to core recommendations for blood pressure measurement, a guideline may state the following: A continuous invasive blood pressure measurement shall be carried out after cardiac surgeries in order to be able to rapidly detect changes in circulation and to carry out routine arterial blood gas analyses. The blood gas analysis is a process for the measurement of the gas distribution or a partial pressure of O₂, oxygen; CO₂, carbon dioxide, as well as a pH value and of an acid-base balance in the blood. In the tool, the modeler may open a mechanism to search the nomenclature of the SDC standard. The modeler may in this case find elements for a continuous invasive blood pressure measurement. The modeler may add the elements to the model.

In addition, the modeler may identify cardiac surgeries from an OPS, Operation and Procedure Key, catalog (5-35 . . . 5-37) and add this to the model The OPS catalog is now the German modification of the International Classification of Procedures in Medicine, ICPM, and the official classification of operational procedures for control of services, proof of services and basis for billing of services of German hospitals and practicing physicians.

As “brief,” a period of 24 hours after completion of the surgery may be defined here as a limitation. Finally, the elements may be linked with conditions in order to be able to check both the presence of a measurement as well as of a procedure, which, for example, a Runtime Engine or the processing device shall consider during the execution.

The delivery in the hospital of the medical knowledge model may be carried out as described below. The digitally signed knowledge model may be installed, for example, in the Runtime Engine. After the installation, the Runtime Engine may check, for example, a release of a certificate preparer by an operator, the validity of the certificate and/or whether the rules in the knowledge model are in conflict with no other models and can be executed. The operator may release certificates of the DGAI. The certificate may be trustworthy and have, for example, a validity until November 2022. A result of the checking of the knowledge model would thus be positive. The knowledge model may become active.

Updates of the knowledge model may be carried out as described below. The DGAI may prepare an updated knowledge model with additional recommendations after a next scheduled check, for example, in November 2022. The update may be provided with a new signature and be ready for use after a publication.

The exemplary embodiment shown in FIG. 3 may comprise one or more additional optional features.

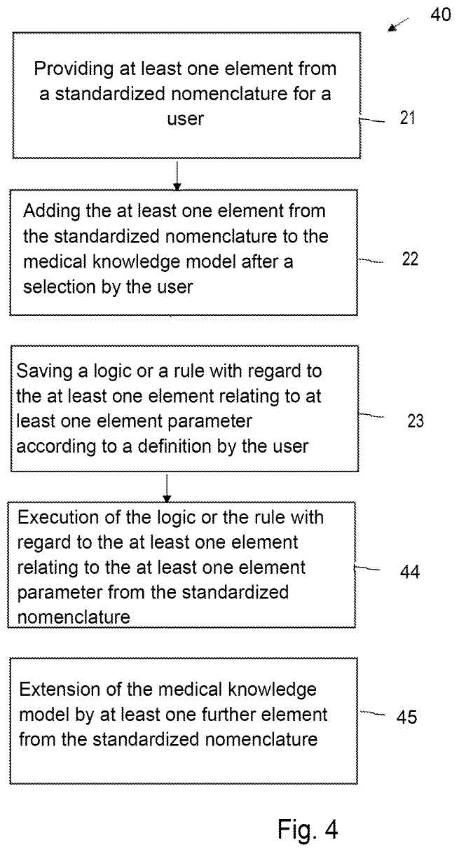

FIG. 4 shows a block diagram of an exemplary embodiment of a process 40 for the formalized application of medical knowledge. The process 40 comprises the process 20, 30 described in conjunction with FIG. 2 and/or FIG. 3. Furthermore, the process 40 may comprise an execution 44 of the logic or the rule with regard to the at least one element concerning the at least one element parameter from the standardized nomenclature. The processing device 11 may, furthermore, in this case generate output information during the execution of the logic or of the rule. The output information may be provided from the one or more interfaces 12. The output information may comprise, for example, an instruction, which pertains to a measurement, the presence of a measurement, and/or a check of a result of a measurement, which pertains, for example, to blood values, respiratory rate, blood pressure, oxygen saturation and/or heart rate of the patient. The process 40 may further comprise an expansion 45 of the medical knowledge model by at least one additional element from the standardized nomenclature. The expansion 45 of the medical knowledge model may also comprise, for example, an updating of the medical knowledge model, in addition to the adding of medical knowledge from a standardized nomenclature. During the expansion 45 of the medical knowledge model, additional logical links, which pertain to the additional element, may also be incorporated into the medical knowledge model.

The exemplary embodiment shown in FIG. 4 may comprise one or more additional optional features.

Functions of different elements shown in the figures as well as the designated function blocks may be implemented in the form of dedicated hardware, e.g., of “a signal provider,” of “a signal processing unit,” of “a processor,” of “a control,” etc. as well as hardware capable of executing software in conjunction with corresponding software. In case of provision by a processor, the functions may be provided by a single dedicated processor, by a single, jointly used processor or by a plurality of individual processors, some of which or all of which can be used jointly. However, the term “processor” or “control” is far from being limited to hardware capable exclusively of executing software, but it may comprise digital signal processor hardware (DSP hardware; DSP=Digital Signal Processor); network processor, application-specific integrated circuit (ASIC=Application Specific Integrated Circuit), field-programmable logic array (FPGA=Field Programmable Gate Array), read-only memory (ROM=Read Only Memory) for storing software, random-access memory (RAM =Random Access Memory) and non-volatile storage device. Other hardware, conventional and/or customer-specific, may also be included.

A block diagram may represent, for example, a schematic circuit diagram, which implements the basic principles of the disclosure. In a similar manner, a flow chart, a flow diagram, a state transition diagram, a pseudocode and the like may represent different processes, operations or steps, which are represented, for example, essentially in computer-readable medium and are thus executed by a computer or processor, regardless of whether such a computer or processor is shown explicitly. Processes disclosed in the specification or in the patent claims may be implemented by a component, which has a means for executing each of the respective steps of these processes.

It is apparent that the disclosure of a plurality of steps, processes, operations or functions, which are disclosed in the specification or in the claims, shall not be considered to be in the defined order, unless this is explicitly or implicitly state otherwise, e.g., for technical reasons. Therefore, these will not be limited to a defined order by the disclosure of a plurality of steps or functions, unless these steps or functions are not replaceable for technical reasons. Further, a single step, function, process or operation may in some examples include a plurality of partial steps, partial functions, partial processes or partial operations and/or be broken up into same. Such partial steps may be included and be a part of the disclosure of this single step, unless they are explicitly ruled out.

While specific embodiments of the invention have been shown and described in detail to illustrate the application of the principles of the invention, it will be understood that the invention may be embodied otherwise without departing from such principles.

### LIST OF REFERENCE NUMBERS

10 Device for generating a medical knowledge model

11 Processing device

12 Interface

20 Process for generating a medical knowledge model

21 Provision of at least one element from a standardized nomenclature for a user

22 Addition of the at least one element from the standardized nomenclature to the medical knowledge model according to a selection of the user

23 Storage of a logic or of a rule with regard to the at least one element concerning at least one element parameter according to a definition of the user

30 Process for generating a medical knowledge model

31 Search of the standardized nomenclature for elements

32 Addition of the element to the medical knowledge model

33 Definition of a logic and criteria based on the elements

34 Automatic validation of the medical knowledge model

35 Addition of a digital signature to the medical knowledge model

36 Provision of the signed medical knowledge model

What is claimed is:

1. A device for generating a medical knowledge model, the device comprising:
a processing device, which is configured:
to provide at least one element from a standardized nomenclature for a user;
to add the at least one element from the standardized nomenclature to the medical knowledge model according to a selection of the user;
to store a logic or of a rule with regard to the at least one element concerning at least one element parameter according to a definition of the user; and
to add a digital signature to the medical knowledge model; and
one or more interfaces that are configured to provide the medical knowledge model, and to receive a user input.

2. A device in accordance with claim 1, wherein the processing device is further configured to validate a consistency of the medical knowledge model.

3. A device in accordance with claim 1, wherein the processing device is further configured to offer to the user at least one element from the group comprising physiological variables, settings, roles, mathematical and statistical operations, Boolean logic, an “if-then” case distinction and temporal criteria as the logic or rule.

4. A device in accordance with claim 1, wherein the at least one element comprises at least one medical element comprising a measurement of a vital parameter of a patient.

5. A medical knowledge model system for the formalized application of medical knowledge, comprising a device for generating a medical knowledge model, the device comprising:
a processing device, which is configured:
to provide at least one element from a standardized nomenclature for a user;
to add the at least one element from the standardized nomenclature to the medical knowledge model according to a selection of the user;
to store a logic or of a rule with regard to the at least one element concerning at least one element parameter according to a definition of the user;
to add a digital signature to the medical knowledge model;
to execute the logic or of the rule with regard to the at least one element concerning the at least one element parameter from the standardized nomenclature; and
to expand the medical knowledge model by at least one additional element from the standardized nomenclature; and
one or more interfaces that are configured to provide the medical knowledge model, to receive a user input and to provide the at least one element.

6. A medical knowledge model system in accordance with claim 5, wherein:
the processing device is further configured to generate output information during the execution of the logic or of the rule; and
the one or more interfaces are further configured to provide the output information.

7. A process comprising the step of generating a medical knowledge model, wherein the step of generating comprises the steps of:
providing at least one element from a standardized nomenclature for a user;
adding the at least one element from the standardized nomenclature to the medical knowledge model according to a selection of the user;
storing a logic or a rule with regard to the at least one element concerning at least one element parameter according to a definition of the user; and
adding a digital signature to the medical knowledge model.

8. A process in accordance with claim 7, further comprising validating a consistency of the medical knowledge model.

9. A process according to claim 7, further comprising providing a formalized application of medical knowledge, comprising:
executing the logic or of the rule with regard to the at least one element concerning the at least one element parameter from the standardized nomenclature, and
expanding the medical knowledge model by at least one additional element from the standardized nomenclature.

10. A process according to claim 7, further comprising providing a computer program with a program code for carrying out at least one of the process steps upon executing the program code on a computer, on a processor or on a programmable hardware component.

11. A process according to claim 9, further comprising providing a computer program with a program code for carrying out at least one of the process steps upon executing the program code on a computer, on a processor or on a programmable hardware component.
